(19) United States
(12) Patent Application Publication
(10) Pub. No.: US 2026/0104413 A1
(43) Pub. Date: Apr. 16, 2026

(54) MULTIPLY LABELLED POLYMERIC CONSTRUCTS FOR DETECTION ASSAYS

(71) Applicant: Cambridge Molecular Diagnostics Ltd, Cambridge (GB)

(72) Inventors: Gustavo Andrés CERDA MOYA, Cambridge (GB); Benjamin Fraser Adam SHAW, Cambridge (GB)

(73) Assignee: Cambridge Molecular Diagnostics Ltd, Cambridge (GB)

(21) Appl. No.: 19/249,832

(22) Filed: Jun. 25, 2025

Related U.S. Application Data
(63) Continuation of application No. 18/280,923.

(30) Foreign Application Priority Data
Mar. 10, 2021 (GB) 2103321.2

(51) Int. Cl. G01N 33/543; G01N 33/58

(52) U.S. Cl. CPC G01N 33/54388; G01N 33/587; G01N 2333/01

(57) ABSTRACT

The present invention relates to methods, kits and devices for detecting a quantity of target molecule. The invention is particularly relevant to techniques carried out on a flow based assay device. Each biological target molecule is a protein capsid decorated with multiple copies of affinity tags and/or multiple copies of protein or peptide binding partners in order to bind a plurality of detectable markers.

### CROSS-REFERENCE TO RELATED APPLICATIONS

This application is a continuation of U.S. patent application Ser. No. 18/280,923 filed Sep. 7, 2023, which is a national phase application under 35 U.S.C. § 371 that claims priority to International Application No. PCT/GB2022/050630 filed Mar. 10, 2022, which claims priority to Great Britain Application No. 2103321.2 filed Mar. 10, 2021, all of which are incorporated herein by reference in their entirety.

### INCORPORATION OF SEQUENCE LISTING

The instant application contains a Sequence Listing, named “SIPM.P0009US.C1_sequence_listing.xml” (created Jun. 25, 2025 and is 149 kilobytes in size) which has been submitted electronically in XML format and is hereby incorporated by reference in its entirety.

### FIELD OF THE INVENTION

The present invention relates to methods, kits and devices for detecting a quantity of particular biomolecules, for example proteins or nucleic acids. The invention is particularly relevant to techniques carried out on a flow based assay device. The presence or absence of the biomolecules may be detected on the device using an optical read-out.

### BACKGROUND TO THE INVENTION

When detecting biomolecules in real-world situations, it is advantageous to accurately detect as few copies of the molecules as possible using simple instrumentation. Whilst nucleic acid samples can be amplified to increase the concentration of particular sequences, antibodies, proteins or other non-nucleic acid biomolecules cannot be amplified, and hence the detection is limited by the number of such molecules appearing in the sample.

Nucleic acid amplification techniques usually require some form of hardware in order to ensure amplification occurs. Often the introduction of additional reagents is also required. Neither of these is desirable in situations where a fast and cheap read out on the presence or absence of a particular biomolecule in a sample is desired.

When performing genetic analysis, there is generally a need to amplify the number of copies in the sample, as the number present in the sample is generally too few to be detected. This can be done using, for example, thermocycling or isothermal amplification. PCR and Isothermal base amplification methods have been developed to allow the detection of low amounts of DNA or RNA. However this method can take about 90 min to generate results.

Thermocycling assays require hardware for heating and cooling as well as a means for detecting the presence of the amplified products.

Isothermal amplification techniques include SDA, LAMP, SMAP, HDA, EXPAR/NEAR, RPA, NASBA, ICAN, SMART. The reaction proceeds at a constant temperature using strand displacement reactions. Amplification can be completed in a single step, by incubating the mixture of samples, primers, DNA polymerase with strand displacement activity, and substrates at a constant temperature. Such methods typically amplify nucleic acid copies at least 109 times in 15-60 minutes. However the requirement remains for a means for detecting the presence of the amplified material. Standard PCR based amplification can detect 1-10 molecules per reaction after 36 cycles (90-120 min). Isothermal amplification can achieve a similar performance to PCR based technologies.

Once the nucleic acid is amplified, a nucleic acid assay requires a secondary detection technology such as spectrophotometry or turbidity. However, such known techniques have drawbacks. Fluorescence detection requires labelling to allow fluorescence, making it expensive. The reagent SYBR green binds to DNA making it inherently carcinogenic; the Ames Test shows it to be both mutagenic and cytotoxic. Also SYBR green is not specific and attaches to any double stranded DNA thus increasing background signal. Turbidity measurements require expensive instrumentation to provide quantification.

In cases where non-nucleic acids are being detected, for example the presence of blood proteins, the sensitivity of the assay is dependent on the number of protein molecules in the sample. The number of molecules of the protein in the sample cannot be increased.

A common molecular detection technology is the lateral flow assay, where molecules are identified via antibody interactions on a support. Lateral flow assays are well known, and have been used for decades in a variety of assay platforms, for example home pregnancy tests. The basic flow assay has been used to develop a plethora of assays for clinical, veterinary, environmental, agricultural, bio-defensive and food-born pathogen screening applications. Strip assays are copiously adaptable and as such are commercially available for an extensive range of analytes including blood protein biomarkers, mycotoxins, viral and bacterial pathogens, as well as a whole range of nucleic acid detection products. However such assays are limited by the amounts of sample required as no amplification is carried out. Such assays have no amplification system, so the assays only work if sufficient amounts of the detected molecules are present in the test solution.

Since their introduction in the 1980s, lateral flow technologies have become important tools for point-of-care and home testing. They are commonly used to detect a broad array of targets such as HcG, infectious diseases and drugs of abuse and are also commonplace in veterinary testing, environmental testing and for monitoring analytes related to the human physiological condition. Initially tests provided a positive/negative result, but the development of reader technology and improvements in the materials and reagents has enabled a progression towards semi-quantitative and quantitative assays.

Lateral flow assays are essentially immunoassays which have been adapted to operate along a single axis to suit the format of a test strip. There are a number of variations of the technology that have been developed into commercial products, but they usually operate using the same basic concept. The technology is based on a series of capillary beds, such as pieces of porous paper or polymer. Each of these elements has the capacity to transport fluid, for example body fluids such as blood, saliva or urine or extracts thereof. A typical lateral flow assay test strip typically consists of the following components:

### 1. Sample Pad

An adsorbent pad onto which the test sample is applied. This acts as a sponge and holds an excess of sample fluid.

### 2. Conjugate or Reagent Pad

Once the sample pad is saturated the fluid migrates to a porous conjugate pad which contains antibodies specific to the target analyte conjugated to coloured particles (usually gold nanoparticles, or latex microspheres but in some instances fluorescent labels are used). When the sample fluid dissipates the matrix, it also dissolves the particles and in one combined, conveying action, the sample and conjugate mix flow through the porous structure. In this way, the analyte binds to the particles while migrating further along the test strip.

### 3. Reaction Membrane

This is typically a membrane onto which anti-target analyte antibodies are immobilised in a stripe that crosses the membrane to act as a capture zone or test line (a control zone will also be present, containing antibodies specific for the conjugate antibodies).

The reaction membrane has one or more areas (stripes) where a third molecule has been immobilized. By the time the sample-conjugate mix reaches these stripes, the analyte has been bound on the particle and the third ‘capture’ molecule in the stripe binds the complex. After a while, when more and more fluid has passed across the stripes, particles accumulate and the stripe-area changes colour. Typically there are at least two stripes: one (the control) that captures any particle and thereby shows that reaction conditions and technology worked fine, the second contains a specific capture molecule and only captures those particles onto which an analyte molecule has been immobilized.

### 4. Wick or Waste Reservoir

A further absorbent pad designed to draw the sample across the reaction membrane by capillary action. After passing the test stripes the fluid enters a final porous material, which simply acts as a waste container.

The components of the test strip are usually fixed to an inert backing material and may be presented in a simple dipstick format or within a plastic casing with a sample port and reaction window showing the capture and control zones.

The sensitivity of the test strip is limited by the amount of material in test solution. One molecule of the target analyte releases one antibody conjugate, which is attached to one detectable coloured particle. The sensitivity of the assay is therefore limited by the concentration of target in the original sample. Such assays are prone to false negative readings if the concentration of the biomolecule in the sample is too low.

### SUMMARY OF THE INVENTION

The present invention relates to methods and devices for improving the detection sensitivity of lateral flow assays. Prior art assays are limited in sensitivity as only one detectable moiety is linked to each sample molecule. The invention herein allows multiple detectable moieties to be captured per individual sample molecule, thereby increasing the level of directly detectable signal.

The concept of amplifying signal via binding of ligands that themselves have multiple binding sites to create branching structures has been used in various techniques. For example EP0354847 FIG. 1. drawing, where AFP is α-fetoprotein, B is biotin, SA is streptavidin and TG is thyroglobin.

The ultimate goal is to create as many sites for the gold conjugate to bind to that will be tethered in the detection zone.

Applicants have developed improved conjugates for use in assays, including lateral flow assays. By producing the structure in a different way to the art above, the applicants can create higher levels of signal amplification than would be possible with the prior art methods. In addition it can be used in more situations as it does not require the antibodies/ligands to undergo further chemical treatment.

The prior art methodology is dependent on use of biotinylated antibodies to amplify the signal (as is the case in patents WO 00/25135 and EP0354847 A2 for example). Essentially antibodies/ligands must be treated with and coated in biotin. This process of NHS-Biotin linking has many downsides. It requires the proteins to be put through a process that can inhibit or reduce their binding function. Furthermore it relies on the availability of reactive groups on the protein surface that are able to react with the chemical linker (NHS-Biotin), which restricts the amount of groups that can be attached without affecting the function of the antibody.

The present disclosure provides improved approaches to signal amplification in assay systems.

A first aspect of the disclosure provides first phage capsids, having a plurality of viral coat proteins, wherein at least a first proportion of the coat proteins comprise an affinity peptide that comprises multiple copies of an affinity tag specific for a binding partner. The binding partner may be bound to a detectable marker, such as a nanoparticle.

The first phage capsid can also comprise a region specific for a target, for example an antibody, coupled to the surface of the capsid. In this way, a single target, when bound by this region, displays multiple copies of the affinity peptide born on the phage capsid. Since the affinity peptide carries multiple copies of the affinity tag, the affinity peptide attracts multiple copies of the detectable marker. This provides significant amplification of the signal.

Further amplification is possible using a second phage capsid. This second capsid is configured to bind the first capsid through tag/binder interactions. The capsid also has a plurality of viral coat proteins, wherein at least a first proportion of the coat proteins carry a binding partner specific for the affinity tags present on the first capsid and a second proportion of the capsid proteins carry an affinity peptide that comprises multiple copies of a second affinity tag. This second affinity tag may be specific for a binding partner that is bound to the detectable marker. In this manner amplification is significantly increased over the first approach as many more detectable markers are associated with the single target. This second capsid provides a further embodiment of the disclosure.

In some embodiments the binding partner of (b) is configured to bind the affinity tag of the phage capsid of claim 2.

The second capsid preferably lacks the region specific for a target.

Of course the affinity tag/binding partner pair can be swapped about in each case so that the first capsid carries the binding partner whilst the marker or the second capsid carries the peptide tags.

The second capsid and the first capsid may be configured to be bound together by interactions between the peptide tags and the binding partners.

Conveniently, since the genome of many phage codes for more than one species of coat protein, the various affinity peptides and binding partners may be expressed as constructs (such as fusions) of separate species of coat protein. In this way, by choosing the appropriate capsid and coat protein, the proportion of one binder or tag to another can be varied.

Described herein is a method of creating fusion (capsid) proteins with multiple subunits of small tags that will then act as docking sites for other specific tags/antibodies (examples include His (HHHHHH), FLAG (DYKDDDDK), E-tag (GAPVPYPDPLEPR), HA (YPYDVPDYA), Strept-tag (WSHPQFEK), Myc (EQKLISEEDL), S-tag (KETAAAKFERQHMDS), SH3 (STVPVAPPRRRRG), G4T (EELLSKNYHLENEVARLKK). Creating these fusion proteins rather than treating the proteins with factors such as biotin has many advantages including but not limited to:
1. It does not require the antibodies to go through further treatments that can inhibit their function
2. It allows control of the location of the tags to not inhibit the function of the antibodies.
3. More labels can be incorporated into each fusion protein without inhibiting function
4. By introducing oligomerization peptides this allows each binding protein subunit to carry 4-7 times the amount of tags. (For example IgG antibody can carry approx. 2-10 biotin molecules. The fusion proteins start with 5-10 repeating subunits and then oligomerize into subunits that each will have 25-70 of the tags. Prior art branches will amplify signal by 2-10x. Each of the proteins described herein will amplify the signal 25-70x.

Also described is a method of increasing the signal available using the capsid structures described herein. Capsids are multi-protein assemblies of self-folding protein structures. A capsid is the protein shell of a virus or bacteriophage, enclosing its genetic material. It consists of oligomeric (repeating) structural subunits made of protein. The proteins making up the capsid are called capsid proteins or viral coat proteins (VCP). Capsids can be found in all bacteriophages, including for example T7, M13.

Any bacteriophage capsid can be engineered as described herein, for example M13, T4, Mu, P1, P2, λ, T5, HK97, N15, T7, T3, φ29 and P22.

Also described herein, a “bacteriophage” or a “phage” is a virus which infects and replicates within a bacteria or archaea. Bacteriophages comprise proteins that encapsulate a DNA or RNA genome. The genomes may encode multiple genes (as few as four genes e.g. MS2 and as many as hundreds of genes). Phages replicate within the bacterium following injection of their genome into its cytoplasm. The bacteriophage structure consists of a capsid head, collar and phage tail.

Also described herein is a first phage capsid comprising a multi protein assembly of capsid proteins, the multi protein assembly comprising a plurality of units, said units being exclusively either:
(i) a peptide comprising repeating amino acid sequences which are affinity tags for specific binding partners; these affinity tags may be configured to bind to specific binding partners on either a second viral capsid or on a detectable nanoparticle; or
(ii) the specific binding partners for said affinity tags. These specific binding partners may be configured to bind to an affinity tag on either a second viral capsid or on a detectable nanoparticle; and

Wherein the first viral capsid additionally comprises a region specific for a target in a sample.

In some embodiments the first phage capsid comprises a multi protein assembly of capsid proteins, the multi protein assembly comprising a plurality of peptides comprising repeating amino acid sequences which are affinity tags for specific binding partners; these affinity tags may be configured to bind to specific binding partners on either a second viral capsid or on a detectable marker; wherein the first phage capsid additionally comprises a region specific for a target in a sample.

Also described herein is a second phage capsid. This second page capsid may be configured to bind the first capsid through tag/binder interactions. The second phage capsid comprising a multi protein assembly of capsid proteins, the multi protein assembly comprising two different units, each units being one of:
(a) capsid proteins that comprises a peptide that comprises at least two repeats of a peptide affinity tag specific for a peptide or protein binding partner; or
(b) capsid proteins that comprise the peptide sequence of a peptide or protein binding partner specific for a peptide affinity tag.

This provides a population of two different types of unit.

The second phage capsid preferably lacks the region specific for a target in a sample.

In some embodiments either the peptide affinity tag is specific for a protein sequence of a peptide or protein binding partner on the first capsid and/or the peptide sequence of a peptide or protein binding partner is specific for a peptide affinity tag on the first capsid, whichever is present.

In some embodiments the phage capsids have two or more of (a) and/or (b) provided that the affinity tags are different in each case. In some embodiments the capsids have at least one of (a) and at least one of (b) or may have at least two of (a) or at least two of (b) for example, particularly one of (a) and one of (b).

Also described herein is a first multiply labelled protein capsid construct comprising a region that is specific for a target and repeating amino acid sequences acting as affinity binding sites that are specific to detectable nanoparticles, the construct having at least two separate affinity binding sites, each affinity binding site configured for linkage to separate detectable nanoparticles.

Also described herein is a second multiply labelled protein capsid construct comprising (i) repeating amino acid sequences acting as affinity binding sites that are specific to detectable nanoparticles, (ii) a plurality of peptide sequences of a peptide or protein binding partner specific for a peptide affinity tag on the first capsid, the construct having at least two separate affinity binding sites, each affinity binding site configured for linkage to separate detectable nanoparticles.

The above two chimeric capsids together also provide an amplification couple which provides levels of amplification superior to that achieved using a single chimeric capsid that comprise a region specific for a target, and multiple copies of an affinity tag.

Also described herein is a multiply labelled protein capsid construct comprising repeating amino acid sequences acting as affinity binding sites that are specific to detectable nanoparticles, the construct having at least two separate affinity binding sites independently linked to separate detectable nanoparticles.

Each capsid can comprise a region that is specific for a target. This arrangement enables a single target molecule to carry a large number of capsids and nanoparticles, thereby improving the sensitivity of detection.

The capsid structure thus contains or comprises multiple protein chains referred to herein as capsid proteins (CP), viral coat proteins (VCP) or protein strands) each chain having multiple labels. The capsid structure allows assembly of the structure such that the affinity binding sites or binding partners are presented on the exterior of the capsid.

In some embodiments, the individual capsid proteins can be expressed having multiple tags, typically in the form of a peptide comprising multiple identical tags (an “affinity peptide”), attached to the N or C terminus, (particularly the C terminus). Alternatively the expression can attach chemically modifiable, or reactive, groups to the N or C terminus, (particularly the C terminus) of capsid proteins, which allows peptides comprising the multiple tags to be chemically bonded to the N or C terminus of the capsid protein, preferably the C-terminus. The same two approaches can be used to provide the peptide or protein binding partner.

Capsids of different types of virus have varying numbers of capsid proteins. Some capsids have multiple species of capsid protein. Typical capsids may have several hundred capsid proteins per capsid. Thus if each capsid protein has 5 to 10 tags, and each capsid has 200-500 capsid proteins, signal amplification of 1000-5000 per capsid are obtained over single labelling.

The capsid can have multiple affinity tags within one or more of the coat proteins. Each capsid protein may comprise 2 or more affinity tags, which typically, may be arranged in series as an affinity peptide. Alternatively the capsid can be engineered to contain or comprise multiple reactive sites enabling chemical attachment of the multiple affinity tags or affinity peptides. The reactive sites may be lysine, or another reactive amino acid such as cysteine, arginine, tryptophan, tyrosine, methionine and/or histidine.

Also described is a method of creating a multiply labelled protein capsid construct, comprising
(i) taking a multi-protein capsid containing a plurality of reactive amino acid subunits;
(ii) chemically linking a plurality of repeating amino acid sequences or affinity tags acting as affinity binding sites that are specific to detectable nanoparticles or to further chimeric capsids, to the reactive amino acid subunits; and optionally
(iii) binding multiple detectable nanoparticles to the affinity binding sites.

The capsid contains multiple capsid proteins wherein two or more of the capsid proteins preferably all the capsid protein or all the capsid proteins of one species of capsid protein, contain the reactive amino acids. The reactive sites may be lysine, or another reactive amino acid such as cysteine, arginine, tryptophan, tyrosine, methionine, histidine. After assembly into the capsid, the capsid can be chemically linked to affinity peptide or other tags. The affinity tags may also be small molecule tags such as biotin. The chemical modification is specific to the surface of the capsid, and does not result in random chemical attachment of tags that results in protein denaturation or loss of function.

The capsid proteins self-assemble to form the capsid structures. The protein capsids can be stored at room temperature for prolonged periods. The capsids can also be lyophilised and kept as dried materials. Unlike other protein structures, the capsids can be rehydrated without significant denaturing or loss of function.

The capsids can be chained together in branched structures. Thus the constructs can comprise two or more capsids. A first capsid may bind to the target, and a second capsid may bind to the nanoparticles. The first capsid may bind to multiple second capsids, and each second capsid to multiple nanoparticles.

Also described herein is a method of creating fusion capsid proteins comprising multiple subunits of repeating amino acids that will then act as sites for chemical linking (eg an affinity peptide as described herein). The linking may attach specific tags and/or antibodies and/or other proteins or peptides as described herein. Creating these fusion proteins with a peptide chain with repeating amino acids as linking sites allows the linking of the protein capsid construct to any existing protein and non-recombinant antibodies (antibodies not produced as capsid protein fusions). Thus a single capsid can be prepared for use in any system to link to any existing protein and non-recombinant antibodies. For example the capsid can be biotin labelled. Alternatively the capsid may be labelled with repeating amino acid binding motifs (eg on an affinity peptide). In either the case of biotin or amino acid motifs, multiple particle labels may be attached to each capsid.

Described herein is a method to detect small numbers of target molecules in a short time using the labelled capsids described herein. Each target molecule in the sample is thus labelled with multiple nanoparticles, thereby increasing the signal obtained from each molecule in the sample.

The multiple binding sites are within or attached to specific sites in the capsid protein rather than randomly attached as appendages such as biotin. Thus the location and number of labels can be accurately designed and controlled. The location allows optimised binding of the labels to maximise the signal obtained.

Also described herein is a method to detect small numbers of target molecules in a short time using labelled capsids as described herein that bind specifically to the target and to either (i) a plurality of detectable markers such as nanoparticles or (ii) to further labelled capsids (as described herein) wherein the further labelled capsids bind specifically to the first labelled capsids and to a plurality of detectable markers.

The technology described herein relates to a polymeric molecular construct comprising a region that is specific for a target, the polymeric molecule having multiple affinity binding sites wherein at least two of the affinity binding sites are configured to bind to a detectable nanoparticle.

The technology described herein further relates to a polymeric molecular construct comprising a region that is specific for a target and a plurality of detectable nanoparticles, the polymeric molecule also having multiple affinity binding sites wherein at least two of the affinity binding sites are each independently linked to a detectable nanoparticle.

The technology described herein further relates to a multi-labelled multi-protein capsid construct comprising a region that is specific for a target and a plurality of detectable nanoparticles, the construct having multiple affinity binding sites wherein at least two of the affinity binding sites are each independently linked to a detectable nanoparticle.

The technology described herein further relates to a multi-labelled multi-protein capsid construct comprising a region that is specific for a target, the construct also comprising multiple affinity binding sites wherein at least two of the affinity binding sites are each independently linkable to a detectable nanoparticle or to a second a multi-labelled multi-protein capsid construct comprising multiple affinity binding sites wherein at least two of the affinity binding sites are each independently linkable to a detectable nanoparticle.

The technology described herein also relates to a flow device for detecting the presence of a target, the device comprising:
(a) a sample loading area positioned at one end of the lateral flow device;
(b) an area comprising a multiply labelled protein capsid construct comprising a region that is specific for a target and repeating amino acid sequences acting as affinity binding sites that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target and capture probes for the polymeric molecular construct, wherein said capture probes are immobilized on the lateral flow device; and
(d) absorbent material, wherein the absorbent material wicks an aqueous sample across the lateral flow device when the aqueous sample is added to the sample loading area.

The technology described herein also relates to a flow device for detecting the presence of a target in a sample, comprising:
(a) a sample loading area positioned at one end of the flow device;
(b) an area comprising a multiply labelled protein capsid construct comprising a region that is specific for a target and repeating amino acid sequences acting as affinity binding sites that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area.

The device can take the form of a test strip where the fluid flow occurs along a single axis. The device may also be referred to as a chip, where the strip is contained within a holder in order to aid handing of the strip. All of the chemicals and reagents required for detection of the target are immobilised onto a solid support surface which is then exposed to the fluid being tested for the target.

In an alternative method, the capsids, multiply labelled polymeric molecular construct or labelled protein construct described herein can be mixed with the sample prior to application to the lateral flow device. In such devices there is no need for the device to contain the area described in (b).

The flow assay can be a lateral flow assay, where fluid flows along a strip of porous material, or a vertical flow assay where the fluid passes through various sections under gravity or capillarity. The vertical and lateral flow can be combined.

The detection can be carried out without the need for any solution reagents as everything required can be immobilised on the surface of the device. Particular applications relate to the identification of biomolecules, for example proteins, antibodies or nucleic acid molecules. No further enzymes or molecules other than those mixed with the sample, or immobilised or adhered to the strip are required. For example the technology allows the detection of RNA without the need for reverse transcriptase, the detection of DNA without the need for polymerase based amplification and the detection of proteins without requiring enzymes or the substrates therefor It therefore allows the detection of small amounts of molecules such as proteins, lipids, saccharides, metabolites, small molecules and chemicals.

The target can be any molecule for which the detection is desired. The target can be a protein. The target can be an antibody. The target can be a lipoprotein. The target can be a small molecule. The target can be a nucleic acid. The target can be DNA, RNA or modified forms thereof. The target may be derived directly from an organism, for example a virus, bacteria or other pathogen. The organism may be mammalian. Where the target is a nucleic acid, the method allows the specific detection of particular sequences, depending on the choice of target specific regions. The target nucleic acid strand may be single stranded or double stranded. In the labelled protein constructs described herein the plurality of regions may be selected from His, FLAG, E-tag, HA, Strept-tag, myc, S-tag, SH3, G4T.

### FIGURES

FIG. 1a: shows an exemplary multiply labelled multi-protein capsid construct as described herein. A capsid construct (A) is labelled with multiple affinity binding sites (X′), each of which are available to bind to a corresponding protein tag (X) or a colloidal gold nanoparticle. One or more capsid constructs may bind together. If two or more capsid constructs bind, each additional capsid construct is shown in FIG. 1b.

FIG. 1b: shows an additional multiply labelled protein capsid construct. The same or different capsid construct (B) is labelled with multiple affinity binding sites (Y′), each of which are available to bind to a corresponding tag (Y) or a colloidal gold nanoparticle.

Thus, each target binding to a single construct A accumulates many colloidal gold nanoparticles. If an additional protein capsid construct which is fully loaded with colloidal gold nanoparticles (FIG. 1b) binds to each X′ of construct A, the signal is amplified when compared to a singly labelled construct A or construct A having colloidal gold nanoparticles bound to each X′.

FIG. 2 shows a lateral flow device for detecting the presence of a target in a sample. The device is printed with two lines on the nitrocellulose membrane (test line and positive control line). The device having a sample loading area (sample pad) with three solutions (2% sucrose, 1% BSA, 0.01% SDS and either (i) Goat-Anti-Human linked T7 phage-400 to 800 G4Ttags, (ii) Anti-GCN4 antibody linked T7phage-400 to 800 SH3 tags, or (iii) Anti-SH3 Colloidal Gold) printed on the cellulose sample pad at positions A1, A2 and A3, respectively. The sample is loaded onto the device at position S and running buffer is added in each position B The device also has an absorbent pad allowing an aqueous sample to be wicked across the lateral flow device.

FIG. 3 shows a control lateral flow device. This device is printed with two lines on the nitrocellulose membrane (test line and positive control line). The device having a sample loading area (sample pad) wherein the sample is loaded at position S. The device also has an absorbent pad allowing an aqueous sample to be wicked across the lateral flow device.

FIG. 4 shows a lateral flow strip of the invention. Constructs of the invention (lanes S and 6) show great sensitivity than lanes 1-4. Nitrocellulose membrane printed strips with 7 lines were used. Top line is 50 ng/μl of pg1-gfp-sh3 molecule, with a log 10 dilution of protein for each subsequent line. The bottom line is 5×10-5 ng/μl. The more lines visible on the strip, the higher the signal amplification. Strips 1 and 2 are control strips, whiles strips 3-6 are results strips. Each strip involves the running buffer run up the strip from the sample pad until it reaches the absorbent pad at the top of the nitrocellulose membrane (NC) strip.

Strip 1 and 2 (controls) run the component anti-biotin up the strip from sample pad.

Strips 3 and 4 (results strips) run both anti-biotin gold and biotinylated MBDIP protein up the strip.

Strips 5 and 6 (results strips) run each of the following components up the strip:
1. T7 bacteriophage with MBDIP protein in gene 10
2. T7 bacteriophage with SH3 protein in gene 10
3. Biotinylated MBDIP protein
4. Anti-biotin gold

The term “labelled capsid” is used herein after to refer generally, to any of “multi-labelled multi-protein capsid construct”, “capsids, having a plurality of viral coat proteins”, “capsid structures” and “viral capsid comprising a multi protein assembly of capsid proteins” as described further herein above.

FIGS. 5A-5J provide a listing of sequence of tags referred to herein, their PDB reference and their binding partners. This list supplements the list provided in the body of the text.

### DETAILED DESCRIPTION OF THE INVENTION

The technology described herein relates to labelled capsid proteins and labelled capsids, to a polymeric molecular construct comprising a plurality of detectable nanoparticles and to lateral flow tests using these. The construct contains multiple protein chains forming a self-assembled capsid, each chain of the capsid being modified to attach multiple labels.

The technology described herein also relates to a labelled capsid comprising a region that is specific for a target and a plurality of regions that are specific to detectable nanoparticles. Each construct has a region that is specific for a target. Each capsid has multiple capsid proteins. Each capsid protein of the multi protein construct has a plurality of detectable nanoparticles. The plurality of nanoparticles can be attached via ligand binding, where suitable amino acid sequences act as affinity binding sites. Thus each labelled capsid has multiple amino acid affinity binding sites within, or attached to, the protein sequence. Each of the multiple affinity binding sites can act independently. Therefore multiple affinity binding sites will act to bind to multiple separate detectable nanoparticles. At least two of the affinity binding sites may be independently linked to a separate detectable nanoparticle, thereby giving a labelled capsid having at least two detectable nanoparticles on the same polymeric molecule. The at least two affinity binding sites may be independently linked to separate detectable nanoparticles.

The use of such constructs means that each individual target molecule will be labelled with multiple nanoparticles, thereby increasing the sensitivity of the detection.

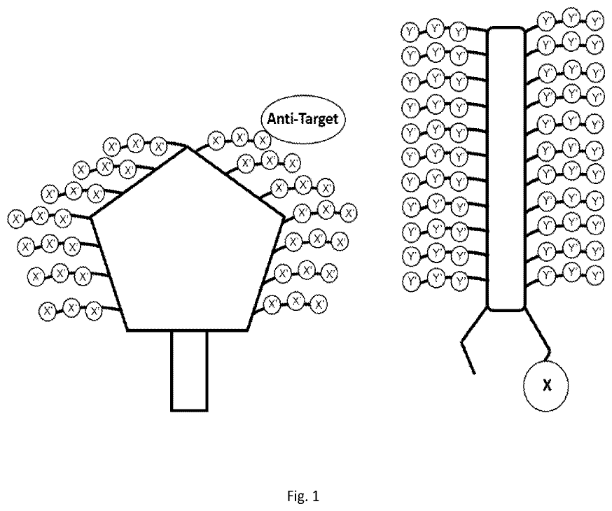

The term comprising is used to indicate that the polymeric molecular construct or labelled protein construct is an assembly of multiple molecular species, and is not necessarily a single covalently linked molecule throughout. Depending on the level of signal amplification, the construct can optionally be a single capsid linked to multiple particles, or can be a single capsid linked to further capsids, where each further capsid can carry the particles (as shown in the FIGS. 1a and 1b combined), thereby obtaining a level of branching which can optionally be repeated using suitable affinity binding regions. Thus the labelled nanoparticles may be bound directly to the capsids, or may be attached via other branched linking groups, for example other capsids or proteins.

For example, FIG. 1a shows an exemplary labelled capsid as described herein. A single target binding moiety (Anti-target) is labelled with multiple receptors (X′) (the amino acid affinity binding sites). The receptors X1 can be linked via chemical attachment or can be part of the integral sequence. The multiple affinity binding sites are available to bind to a moiety (X) which may contain the gold particles. In FIG. 1, three X-moieties are shown per amino acid strand. Multiple strands are assembled as the capsid structure Each triple repeated X′ moiety is attached to the terminus of the capsid proteins, and thus assembles on the exterior surface of the capsid.

Optionally further as seen in FIG. 1b, each X′ can bind to a second capsid having further receptors (Y′). A large number of second capsids can be attached to the first capsid. Each Y′ can bind to Y, Y being attached to a gold particle. Alternatively receptors Y′ can bind to Y, each Y being attached to further multiple receptors Z′. Each Z′ can bind to a colloidal gold particle. Thus each anti-target accumulates many colloidal gold particles via a plurality of binding events. Whilst each colloidal gold particle may bind to many copies of X′ or Y′ per particle, each capsid will also bind to many colloidal particles. When the capsids are constructed in sequential order, the signal is amplified when compared to a singly labelled target (e.g. up to hundreds or thousands of times).

The detectable nanoparticles may be for example antibody coated. The polymeric molecule or labelled protein may contain a plurality of antigens such that each polymeric molecule or labelled protein can bind to more than one nanoparticles (i.e. the affinity sites can be antigens specific to particularly coated nanoparticles). The antigens can be for example peptide regions which can appear repeatedly in particular polymers or proteins. Thus for example the polymers or proteins can be repeating strings of particular amino acids where the amino acids act as antigens (receptors) to antibodies coated on the nanoparticles. Suitable particles may include for example gold-antibiotin or anti-SH3 colloidal gold.

In one embodiment, the labelled capsid comprises:
(a) a region specific for a target from a sample;
(b) multiple amino acid affinity binding sites each configured to bind detectable markers such as nanoparticles, wherein said detectable nanoparticles are antibody coated and said antibodies are linked to the affinity binding sites of the capsid such that each capsid is linked to a plurality of detectable nanoparticles.

In one embodiment, the labelled capsid comprises:
(a) a region specific for a target from a sample;
(b) multiple first amino acid affinity binding sites for a binding species having further affinity binding sites which may be the same or different;
(c) more than one binding species configured to attach to first affinity sites in the form of further capsids, which further capsids comprise further affinity binding sites of the binding species
(d) a plurality of detectable nanoparticles, wherein said detectable nanoparticles are antibody coated and said antibodies are configured to be linked to the further affinity binding sites of the binding species on the further capsids such that each of the multiple binding species is linked to a plurality of detectable nanoparticles, and each construct is in a branched configuration.

There is no upper limit on the level of signal obtained using capsid to capsid assembly. In one embodiment, the system binds between 100 and 10,000 detectable nanoparticles per capsid. In one embodiment, the system binds at least 500 detectable nanoparticles. In one embodiment, the polymeric molecular construct, capsid or labelled protein construct has between 1000 and 10000 detectable nanoparticles.

Phage capsids may comprise two or more types, or species of capsid protein. In some embodiments a first species of capsid protein comprises the affinity tags whilst a second species of capsid protein comprises binding partners. Alternatively a first species of capsid protein comprises one of (i) the affinity tags and (il) the binding partners, whilst the second species of capsid protein comprises the reactive amino acids, which may be modified as described further herein such as to comprise the other of to provide the other of the affinity tags and the binding partners.

Detectable markers can include any marker used in visualisation of targets in ELIZAs, Western blots, lateral flow tests and similar systems. To that end the marker can be a fluorescent marker, an enzyme. A fluorescent protein, a radiological marker and so on, but is typically a nanoparticle or a colloidal nanoparticle.

In one embodiment such nanoparticles are selected from: gold, iron, copper, silver, silver nucleated gold, platinum, carbon and cellulose nanoparticles. In a particular embodiment the detectable marker is a gold nanoparticle or a colloidal gold nano particle.

The nanoparticles are typically sub-micrometer in size. Typically nanoparticles can be 5-100 nanometers in size. The shape of the particles is unimportant, although the particles are often spherical, but can be cylindrical or any other shape, thus size is measured across the largest distance. A larger increase in signal can be obtained from smaller nanoparticles. In some embodiments the size of the particles may be between 5 and 20 nm. The average size of the particles may be 5, 10, 15 or 20 nm for example.

In order to couple the detectable marker to the capsid it is modified by coupling to it either tags or binding partners as described above such that the marker can bind to a capsid bearing either tags or binding partners. Typically the marker will be bound to antibodies, or binding fragments thereof, or to a non-antibody protein or peptide binding partner as described elsewhere herein.

The term affinity binding site or tag, refers to a region of amino acid/peptide sequence. These affinity tags are typically peptide sequences that are specifically recognised by binding partners, which are typically proteins or peptides (including antibodies or binding fragments thereof) The affinity binding tag can be a region of amino acid/peptide sequences specific to a particular antibody or binding fragment thereof (such tags are referred to as epitope tags) or it can be sequence that is recognised by a specific non antibody based peptide or protein binding partner. In some cases the non-antibody based peptide or protein binding partner may form a covalent bond with the tag sequence. The affinity tags and proteins may therefore be provided as part of a chimeric capsid protein.

In some embodiments, the affinity binding tag can be one or more of Glu-Glu-tag, HA-tag, Myc-tag, GCN4-tag, Anti-SH3 protein, ubiquitin. In the polymeric molecular constructs or labelled protein constructs described herein the plurality of regions can be selected from His, FLAG, E-tag HA, Strept-tag, myc, S-tag, SH3, G4T, MbDIP.

The individual affinity tags may be arranged as concatamers as part of an “affinity peptide” comprising multiple affinity tags, the C-terminal of one being directly connected to the N-terminal of the next. Alternatively the tags may be separated by spacer peptides. The spacers may be from 1 to 20 amino acids long, for example from 2 to 10 or 2-4 long. In one example such spacers comprise repeating serine-glycine units or glycine-serine units. One example of such spacers is SGSG, or SGSGSG. Such spacers may also be used between the affinity peptide and the capsid protein in the chimeric capsid proteins. The affinity peptide comprises at least two affinity tags.

In some embodiments the affinity peptide comprises up to 3, 4, 5, 6, 7, 8, 9, 10 or more tags. Each tag on the affinity peptide is the same.

A non-limiting list of example peptide affinity tags given is below. Antibodies to many of these epitope tags are available commercially, see for example those labelled with *:

Epitope tags
*Alfa-tag (SRLEEELRRRLTE)
C-tag (EPEA)
Calmodulin-tag (KRRWKKNFIAVSAANRFKKISSSGAL)
*E-tag (GAPVPYPDPLEPR)
*FLAG (DYKDDDDK)
G4T (EELLSKNYHLENEVARLKK) recognised by Anti-GCN4 antibody
Glu-Glu tag (EEEEYMPE)
HA (YPYDVPDYA)
*Myc (EQKLISEEDL)
NE-Tag (TKENPRSNQEESYDDNES)
Rho1D4-tag (TETSQVAPA)
Softag 1 (SLAELLNAGLGGS)
Softag 3 (TQDPSRVG)
Spot-tag (PDRVRAVSHWSS)
**S-tag (KETAAAKFERQHMDS)
*T7tag (MASMTGGQQMG)
RQLINTNGSWHIN
PDRKAAVSHW
TC-tag (Ty-1 tag) (EVHTNQDPLD)
*VSV-tag (YTDIEMNRLGK)
Xpress-tag (DLYDDDDK)
Non antibody protein or peptide binding tags
*Avi-tag (GLNDIFEAQKIEWHE) (binds to Strepavidin but also Abs availble)
Dog-tag (DIPATYEFTDGKHYITNEPIPPK) binds to DogCatchertm affinity peptide
Isopep-tag (TDKDMTITFTNKKDAE) forms isopeptide bonds to its binding partner, a modified
pilin C
SBP-tag (MDEKTTGWRGGHVVEGLAGELEQLRARLEHHPQGQREP) binds strepavidin
Sdy-tag (DPIVMIDNDKPIT) binds to Sdy catcher
SH3-tag (STVPVAPPRRRRG) binds to proteins with XPXXP concensus and to MbDIP
Snoop-tag ™ (KLGDIEFIKVNK) binds to Snoopcatcher ™
Spy-tagtm (AHIVMVDAYKPTK) binds to Spycatcher ™ protein
**Strep-tag (WSHPQFEK) streptavidin and antibodies
MbDIP
(GRLDLPPGFMFKVQAQHDYTATDTDELQLKAGDVVLVIPFQNPEEQDEGWLMGVKESDW
NQHKELEKCRGVFPENFTERVP) binds to SH3
Charge based affinity
Poly Glutamate-tag (EEEEEEE)
Poly Arginine-tag (RRRRRRR)
*His (HHHHHH)
Ty-tag (CCPGCC) prob not requires a bi arsenide compound
Other epitope and binding domain tags
VDAVN
RRRETQV
GMRPPPPGIRG
APPLPPRN
ANSRFPTSII
PWATCDS
DAEFRHDS
PTSSEQI
ENQKEYFF
LELDKWASL
DKQVEYLDLDLD
ARTKQTARKST
PKLEPWKHP
ITIPVTFE
SDATEGHDED
TPEAPPCYMDVI
RTFRQVQSSISDFYD
VDAVN
RRRETQV
GMRPPPPGIRG
APPLPPRN
ANSRFPTSII
PWATCDS
DAEFRHDS
PTSSEQI
ENQKEYFF
LELDKWASL
DKQVEYLDLDLD
ARTKQTARKST
PKLEPWKHP
ITIPVTFE
SDATEGHDED
TPEAPPCYMDVI
RTFRQVQSSISDFYD
*available from GenScript Biotech (Netherlands) B.V.
**available from BioRad laboratories Watford UK

The binding species or binding partner can be any species corresponding to the above-mentioned binding tags, in other words any species, particularly a peptide or protein (including antibodies or binding fragments thereof), that specifically bind the tag. These are exogenous sequences, ie they are not present in wild type phage or in strains used to prepare the capsids.

In some embodiments the binding species or binding partner can be an antibody or an epitope binding fragment thereof. Such an approach is suitable generally for peptide sequence tags, but is particularly suitable for peptide tags that are designed as epitope tags, as suitable antibodies or fragments are available commercially. Antibody binding species include, but are not limited to Anti-Glu-Glu-tag, Anti-HA antibody, Anti-Flag antibody, Anti-Myc antibody, anti-GCN4 antibody. Anti-SH3 protein. Further examples are provided above.

In some embodiments the peptide tag is specific for a non-antibody peptide or protein binding partner. One example of such a binder-tag pair is MbDIP which binds the SH3 tag other examples of such binder/tag pairs include but are not limited to, those recited above.

In some embodiments the binder can be a ubiquitin binding protein.

In some embodiments the tag can be biotin.

Reference to antibodies includes reference to epitope binding fragments thereof. Such fragments include without limitation: Half-IgGs (product of selectively reducing disulphide bond in the hinge) F(ab′)2 fragments, Fab′ fragments, Fab fragments and Fv fragments.

The target can be any molecule for which the detection is desired. In non-limiting examples, the target can be a nucleic acid, for example DNA, RNA or modified forms thereof. The target can be a particular protein, peptide or antibody, a lipoprotein or polysaccharide. The target may be a drug or a drug metabolite. The target may be a metabolic intermediate.

The target may be derived directly from an organism, for example a virus, bacteria or other pathogen. The target may be from a eukaryotic source, a microorganism, a virus, or a microbiome. The source may be mammalian. The target may be a particular sequence of nucleic acid, DNA, RNA or protein. The target nucleic acid may be single stranded or double stranded.

In some embodiments the region specific for a target can be a nucleotide sequence, which is complementary or partially complementary to a DNA sequence target, such as for a DNA or RNA target. In some embodiments the region specific for a target can be an antibody or a target binding fragment thereof.

In some embodiments the region specific for a target can be a non-antibody protein which is specific for its cognate binding partner, where the cognate binding partner is the target, or it can be the cognate binding partner, where the non-antibody protein is the target.

In one embodiment, the eukaryotic source is selected from algae, protozoa, fungi, slime molds and/or mammalian cells. In one embodiment, the microorganism or virus is selected from Escherichia, Campylobacter, Clostridium difficile, Enterotoxigenic E. coli (ETEC), Enteroaggregative Escherichia coli (EAggEC), Shiga-like Toxin producing E. coli, Salmonella, Shigella, Vibrio cholera, Yersinia enterocolitica, Adenovirus, Norovirus, Rotavirus A, Cryptosporidium parvum, Entamoeba histolytica, Giardia lamblia, Clostridia, Methicillin-resistant Staphylococcus aureus MRSA, Klebsiella pneumonia, flu, Zika, dengue, chikungunya, West Nile virus, Japanese encephalitis, malaria, HIV, H1N1, and Clostridium difficile resistant organisms.

In one embodiment, the sample is a biological sample from a subject. The biological sample from the subject can be selected from stool, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool water, pancreatic juice, lavage fluids from sinus cavities, bronchopulmonary aspirates, blastocyl cavity fluid, and umbilical cord blood.

Amplification couples are combinations of a first labelled capsid comprising a region specific for a target and another capsid leading to a further amplification of signal over that of labelled capsid comprising a region specific for a target alone. In the amplification couple the first labelled capsid is linked to a second labelled capsid through a binding tag/binding partner association.

The disclosure therefor also provides amplification couples comprising any labelled capsid comprising a region specific for a target (the first labelled capsid) as described herein, linked to a further labelled capsid, (the second labelled capsid-which preferably lacks the region specific for a target) as described herein through a binding tag/binding partner association. Wherein the further labelled capsid comprises further un bound binding tags or binding partners. Preferably these binding tag and partners are different to those involved in the binding of the first labelled capsid to the second labelled capsid. These unbound tags and partners are then available to bind a detectable marker comprising a cognate tag or partner as appropriate.

Signal couples are combinations of any labelled capsid and a detectable marker where the detectable marker is bound to any labelled capsid through a binding tag/binding partner association.

The technology described herein also provides flow devices for detecting the presence of a target, the device comprising:
(a) a sample loading area,
(b1) an area comprising a first labelled capsid comprising a region that is specific for a target as described herein, wherein the labelled capsid is capable of wicking across at least a portion of the lateral flow device;
(c) an area specific for the detectable marker;
(d) an area comprising capture probes for the specific target, and optionally a separate area comprising capture probes specific for the labelled capsid; wherein said capture probes are immobilized on the lateral flow device; and
(d) absorbent material, wherein the absorbent material wicks an aqueous sample across the lateral flow device when the aqueous sample is added to the sample loading area.

In some embodiments, the flow device additionally comprises a further area (b2) comprising a second labelled capsid as described herein, which is configured to bind the first capsid through tag/binder interactions wherein the second labelled capsid is capable of wicking across at least a portion of the lateral flow device;

In some embodiments, the flow device comprises a solid support and a lateral flow assay test strip. The solid support can be selected from glass, paper, nitrocellulose and thread. The lateral flow assay test strip consist of the following components:

An adsorbent pad onto which the test sample is applied. This acts as a sponge and holds an excess of sample fluid.

A porous reagent pad comprising:
(i) a first labelled capsid comprising a region specific for the target as described elsewhere herein;
(ii) a detectable marker as described elsewhere herein; and optionally
(iii) a second labelled capsid; wherein the second labelled capsid is configured to bind the first labelled capsid through tag/binder interactions.

Once the sample pad is saturated the fluid migrates to the reagent pad. When the sample fluid dissipates the matrix, it also dissolves the labelled capsids and in one combined, conveying action, the sample, the capsid mix and the detectable marker flow through the porous structure. In this way, the analyte binds to the first capsid, which binds the detectable marker while migrating further along the test strip.

A reaction membrane. This is typically a membrane onto which anti-target analytes are immobilised in a stripe that crosses the membrane to act as a capture zone or test line (a control zone can also be present, for example containing analytes specific for a capsid).

After a while, when more and more fluid has passed across the stripes, detectable markers accumulate and the stripe-area changes colour. Typically there are at least two stripes: one (the control) that captures any particle and thereby shows that reaction conditions and technology worked fine, the second contains a specific capture molecule and only captures those particles onto which an analyte molecule has been immobilized.

A wick or waste reservoir.

A further absorbent pad designed to draw the sample across the reaction membrane by capillary action.

After passing the test stripes the fluid enters a final porous material, which simply acts as a waste container. The components of the test strip may be fixed to an inert backing material and may be presented in a simple dipstick format or within a plastic casing with a sample port and reaction window showing the capture and control zones.

In some embodiments, the flow device can take the form of a test strip where the fluid flow occurs along a single axis. The device may also be referred to as a chip, where the strip is contained within a holder in order to aid handing of the strip. All of the chemicals and reagents required for detection of the target are immobilised onto a solid support surface which is then exposed to the fluid being tested for the target.

In some embodiments, the flow device can be a lateral flow device, where fluid flows along a strip of porous material, or a vertical flow assay where the fluid passes through various sections under gravity or capillarity. The vertical and lateral flow can be combined.

In some embodiments, detection can be carried out without the need for any solution reagents as everything required can be immobilised on the surface of the device.

Particular applications relate to the identification of protein or nucleic acid molecules. No further enzymes or molecules other than those immobilised are required. For example the technology allows the detection of RNA without the need for reverse transcriptase or the detection of DNA without the need for polymerase based amplification. It also allows the detection of small amounts of other molecules such as lipoproteins, lipids, saccharides, polysaccharides, metabolites, small molecules and chemicals.

In one embodiment, the measurement may be a simple end point detection (is the target present; yes or no), or may involve an element of quantitative analysis. For quantitative analysis, the device can be coupled to a suitable reader allowing a direct measurement of the signal intensity in the detection zone. This can be correlated to the number of target molecules present in the target sample. For semi-quantitative analysis, the detection zone can be calibrated to bind different amounts of detectable marker, such as coloured particles (for example gold colloidal stained protein, bound to a printed antibody).

For end point or semi-quantitative detection, the detection can be carried out using the human eye, rather than requiring any further hardware to read the result.

In one embodiment, detection can be carried out in multiple zones or lines. For example, for semi quantitative detection different lines with different amount of trapping molecules can be printed (e.g. a first line containing 25 ng/cm, a second line containing 250 ng/cm, a third line containing 2.5 g/cm and so on). Therefore, considering the molecular weight of the trapping molecule, the accumulation of colour on the stripes will be a reflection of the amount of target on the sample (i.e. if sample contains 1-10 target molecules, only first line will accumulate the colour. If sample contains 10-100 target molecules, first and second lines will accumulate the colour. If sample contains 100-1,000 target molecules, first, second and third lines will accumulate the colour etc. Thus the quantification can be carried out using the different bands where the different bands have different responses depending on the amount of detectable marker in the fluid.

Described herein is also a method for detecting the presence of a target in a sample. The sample may be a biological sample and may, for example be from a subject, the subject may be a human or a domestic or farm animal; or the biological sample may be from another source, the method comprising:
i) adding the sample to a sample loading area of a flow device, wherein said device comprises:
(a) a sample loading area;
(b1) an area comprising a first labelled capsid comprising a region that is specific for a target as described herein, wherein the construct is capable of wicking across at least a portion of the flow device;
(c) an area comprising capture probes for the specific target and capture probes for the labelled capsid, wherein said capture probes are immobilized on the lateral flow device; and (d) absorbent material, wherein the absorbent material wicks an aqueous sample across the lateral flow device when the aqueous sample is added to the sample loading area.
ii) determining from the presence of detectably labelled probe in (c) the presence of the target in the sample.

In some embodiments, the flow device additionally comprises a further area (b2) comprising a second labelled capsid as described herein, which is configured to bind the first capsid through tag/binder interactions wherein the second labelled capsid is capable of wicking across at least a portion of the lateral flow device.

In one embodiment, the present technology can be used for fast detection of the presence of many targets, for example interleukins, hormones, oncogenes (as protein or nucleic acid), pathogens (as protein or nucleic acid), virus (as protein or nucleic acid), drugs, toxins, metabolites. The target can be mycoplasma, for example for identification of cell line contamination. The target can be a coronavirus. Assays and conjugates as described herein can be used for detection of virus infection in mammalian samples.

Fields in which the technology may be used include pathogen identification and contaminant tracing; forensic analyses; food industry; soil analyses; agriculture; aquaculture etc.

Also disclosed is a kit of reagents for detecting a target, the kit containing a device as described above and a buffer solution into which a biological sample can be added. The kit may further include instructions for use of the kit. The device and buffer may be provided sterile. The device may be provided sterile in a closed and sealed packaging.

The invention will now be illustrated by non-limiting examples. Further embodiments of the invention will occur to the reader in light of these.

### EXAMPLES

### Example 1. General Method for Preparation of T7 Bacteriophage Capsids Carrying Affinity Tags and Anti Analyte Antibodies. (FIG. 1a)

This approach targets 400-2000x signal amplification.

The coding sequence for the T7 bacteriophage Gene 10 was cloned into an ampicillin resistant bacterial expression vector under the control of lactose promoter. A sequence of 2 to five affinity tags were cloned in frame of gene 10 at the C terminus.

Knockout T7 bacteriophage for the gene 10 was grown on BL21 E. coli carrying the plasmid for gene 10. Gene 10 expression was induced with IPTG 1 hour before infecting the cells with the knockout phage T7. The bacterial culture was grown over night at 33′° C. and sodium chloride was then added to a final concentration of 1M. Bacterial debris was removed by centrifugation (20 min at 4000g at 4° C.). The bacteriophage were isolated from the supernatant by adding Polyethylene glycol 8000 at final concentration of 10%. The mixture was incubated on ice for 2 hours before precipitating the bacteriophage by centrifugation at 4° C. for 30 min at 4000g. The pellet containing the bacteriophage was resuspended in PBS with sodium chloride at final concentration of 500 mM.

About 1 to 2 amino groups on the bacteriophage were transformed into maleimide groups by reacting with the crosslinker SMCC (Succinimidyl trans-4-(N-maleimidylmethyl) cyclohexane-1-carboxylate) using 1:2 molar ratio.

Thiolation of antibody was performed using SPDP (Succinimidyl 3-(2-pyridyldithio) propionate). The thiol modified antibody was reduced using TCEP or dithiothreitol (DTT) before being added drop by drop to the maleimide modified bacteriophage.

### Example 2. General Method for the Preparation of M13 Bacteriophage Carrying a Protein Tag-Binding Moiety and Tagged with Peptides Carrying 5-10 Tags. (FIG. 1b)

This is an example of a second labelled capsid for use in further enhanced detection. This approach targets 2000-10,000 fold signal amplification, when the second capsid is coupled to the first capsid.

The coding sequence for the M13 bacteriophage gene 3 was cloned into an ampicillin resistant bacterial expression vector under the control of lactose promoter. A non-antibody protein tag-binding partner was cloned into the bacteriophage M13 gene 3 in frame between the leader sequence MKKLLFAIPLVVPFYSHS and alanine 19.

Coding sequence for the M13 bacteriophage gene 8 was cloned into a kanamycin resistance-baring bacterial expression vector under the control of lactose promoter. Two lysines were cloned in frame with the bacteriophage M13 gene 8 after the leader peptide between Alanine 24 and Glutamine 25.

Gene 3 and Gene 8 double knockout M13 bacteriophage were grown on F-factor positive E. coli carrying the plasmid for G8 and G3. G3 and G8 expression was induced with IPTG 1 hour before the infection the bacteria with the knock out bacteriophage M13. The bacterial culture was grown overnight at 33° C. and cells were removed by centrifugation for 20 min at 4000g. M13 bacteriophage were isolated from the supernatant by adding Polyethylene glycol 8000 and sodium chloride at final concentration of 5% and 500 mM respectively. The mixture was stored at 4° C. overnight before precipitating the bacteriophage by centrifugation at 4° C. for 30 min at 4000g. The pellet containing the bacteriophage was resuspended in PBS.

About 2000-3000 amino groups on the bacteriophage were transformed into maleimide groups by reacting the phage with the crosslinker SMCC (Succinimidyl trans-4-(N-maleimidylmethyl)cyclohexane-1-carboxylate) using 1:3000 molar ratio.

Thiolation of affinity peptides comprising 5 to 10 affinity tag sequences was performed using SPDP (Succinimidyl 3-(2-pyridyldithio) propionate). Thiol modified peptides comprising the tags were reduced using TCEP or DTT before they were added drop by drop to maleimide derived bacteriophages. This approach can also be used to couple antibodies specific to particular affinity tags to the phage capsid.

### Example 3. General Method for Preparation of Bacteriophage T7 Chimeric Tag-Binder Phage

This is also an example of a second labelled capsid for use in further enhanced detection.

The coding sequence for the T7 bacteriophage Gene 10 was cloned into an ampicillin resistant bacterial expression vector under the control of the lactose promoter. One to five tags, separated by a GSGS linker, were cloned, in frame with gene 10 at the C terminal (e.g. myc-tag [GSGS-EQKLISEEDL]). The coding sequence for the T7 bacteriophage Gene 10 was cloned into a Kanamycin resistant bacterial expression vector under the control of the lactose promoter. A tag-binding protein (e.g. MbDIP, protein binding partner of the SH3 tag) was cloned in frame with gene 10 at the C terminal using a Serine-Glycine-Serine-Glycine linker between gene 10 and the tag-binding protein.

Knockout T7 bacteriophage for the gene 10 was grown BL21 E. coli carrying both the plasmid coding for the gene 10-tag and the plasmid coding for the gene 10-binder fusion. Gene 10-tag and G10-binder expression were induced with IPTG 1 hour before infecting the cells with the knock out phage T7 at bacterial OD 0.4-0.8.

The bacterial culture was grown for between 3 hours and overnight at 28-33° C., sodium chloride was added to the culture to the final concentration of 1M and bacteria debris were removed by centrifugation (20 min at 4000g at 4° C.). The bacteriophage were isolated from the supernatant by adding polyethylene glycol 8000 at final concentration of 10%. The mixture was incubated on ice for 2 hours before separating the phage by centrifugation at 4° C. for 30 min at 4000g. The pellet containing the phage was finally resuspended in PBS (NaCl 500 mM). These phage particles carry a mixture of tag and binder.

### Example 4. Preparation of Colloidal Gold Protein Conjugates

Colloidal gold solution was prepared following method from Turkevich et al (Discuss. Faraday Soc., 1951, 11, 55-75) and G. Frens (Phys. Sci., 1973, 241, 20-22). 200 mL of 0.01% HAuCl4 in Milli-Q water was stirred vigorously and heated till boiling under reflux conditions. Depending on designated particle size, sodium citrate (1% aqueous solution) was added very quickly while the solution was stirred and boiled vigorously. After about 1 minute the light yellow solution loses colour completely before changing colour to deep blue and finally dark red. To make sure the reaction had finished completely, the solution was allowed to boil for another 10 min. After cooling to room temperature pH was adjusted with 1 ml of 02 M K2CO3 solution. The desired conjugate protein, such as Anti-SH3 protein, was diluted in 10 mL of PBS, and added in small volumes to 200 ml colloidal gold with continuous stirring After mixing the conjugation reaction was left for 30 minutes at room temperature. The appropriate amount of conjugate protein, such as Anti-SH3 protein, was empirically calculated by performing a series dilution and measuring the protection of colloidal gold to 10% Sodium Chloride. After conjugation Anti-SH3 Colloidal gold solution was precipitated at 8000g for 30 min and resuspended in 20 ml of PBS 0.002% Tween-20.

### Example 5. Lateral Flow Test for Detection of PSA Antibodies

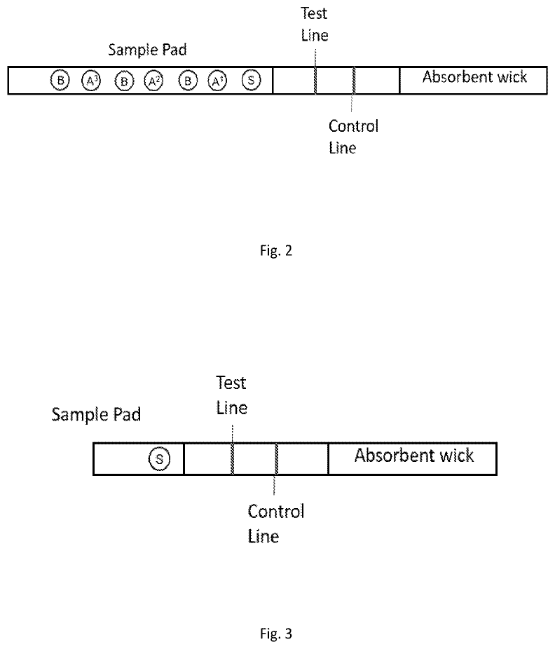

A lateral flow device was printed with two lines on the nitrocellulose membrane (Test Line using 50 ng/cm of human PSA antigen and positive control using 0.5 pg/cm of protein G) (FIG. 3). Three solutions containing 2% sucrose, 1% BSA, 0.01% SDS and either:
(i) Goat-Anti-Human linked T7 phage-carrying 400 to 800 G4T tags,
(ii) Anti-GCN4 antibody linked T7 phage-carrying 400 to 800 SH3 tags; or
(iii) Anti-SH3 Colloidal Gold,
   were prepared, the solutions were printed on the cellulose sample pad on position A¹, A² and A3 respectively (FIG. 2), lateral flow strips where dried for 2 hours at 37° C.

30 μL of blood serum was mixed with 20 μL PBT running buffer (PBS 1% tween), 40 μL of sample mix was loaded on the lateral flow strip (position S FIG. 2), subsequently 40 μL of running buffer (PBS with 0.5% Tween-20) were added in each position B. The presence of PSA antibodies is detected by the accumulation of colloidal Gold at the test line.

Alternatively, 40 μL of sample mix was loaded on the lateral flow device (position S FIG. 2), the device has been previously printed on the nitrocellulose membrane (Test Line using 50 ng/cm of human PSA antigen and Positive control using 0.5 pg/cm of protein G) (FIG. 3). Subsequently, 40 μl of PBT with 1 ng/μl Goat-Anti-Human linked T7phage-400 to 800 G4Ttags, followed by 40 μl of PBT with 1 ng/μl Anti-GCN4 antibody linked T7phage-400 to 800 SH3 tags. And finally 40 μl of PBT of OD 0.1 of Anti-SH3 Colloidal Gold.

### Example 6. Comparison of Signal Amplification

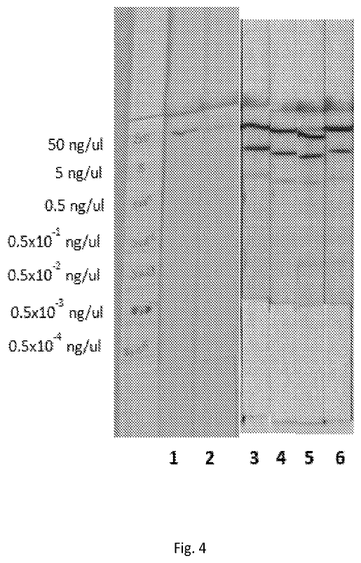

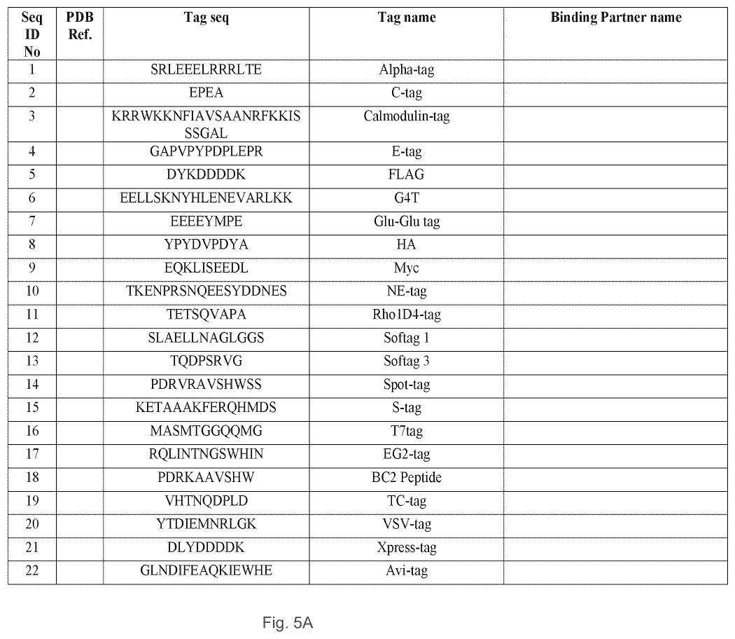

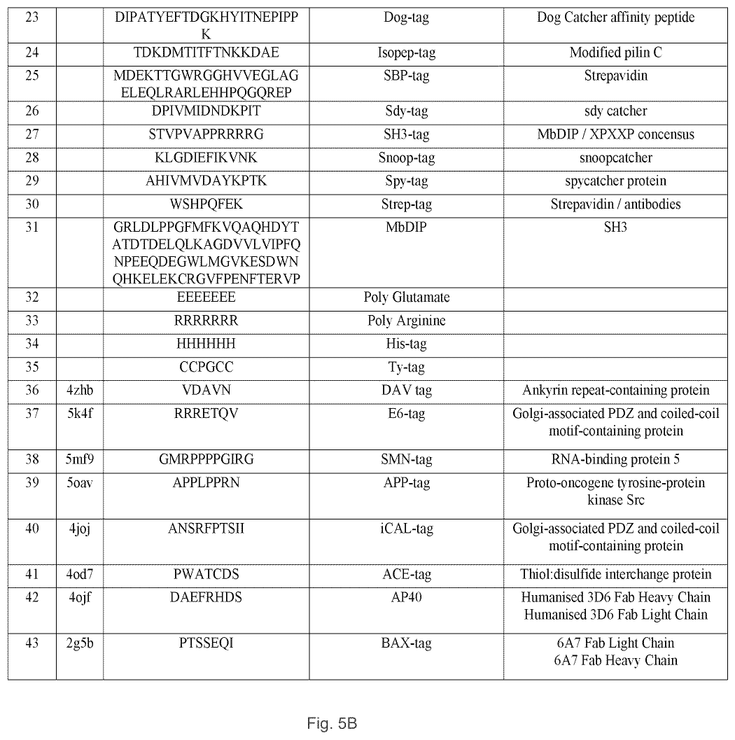

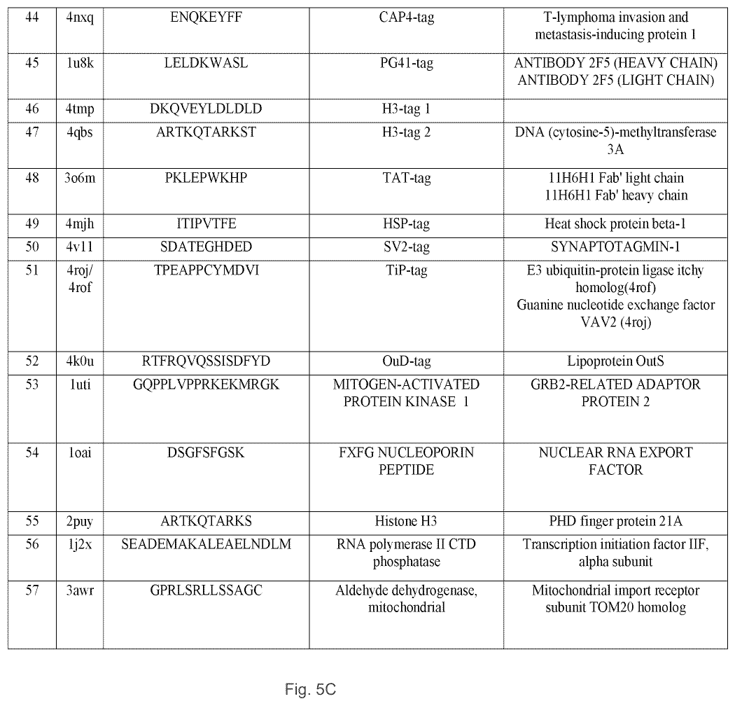

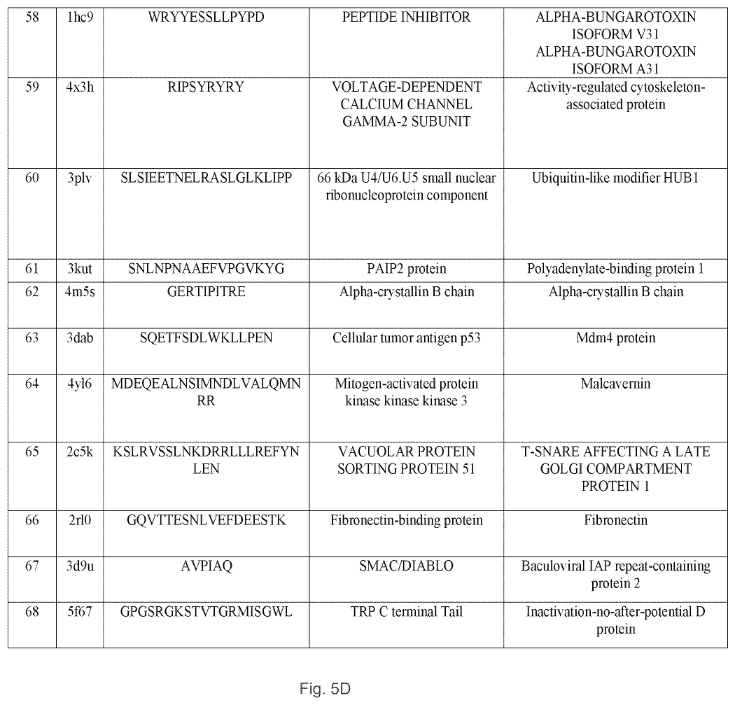

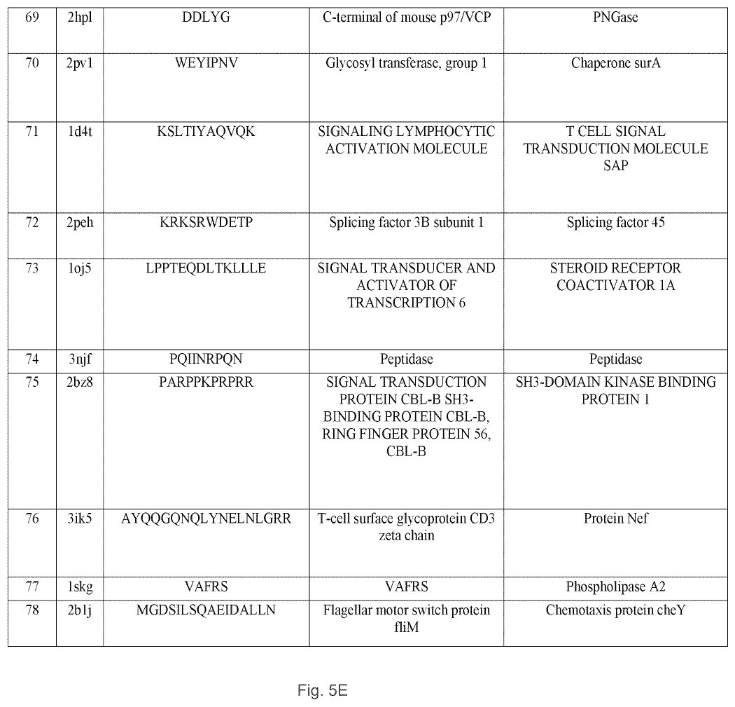

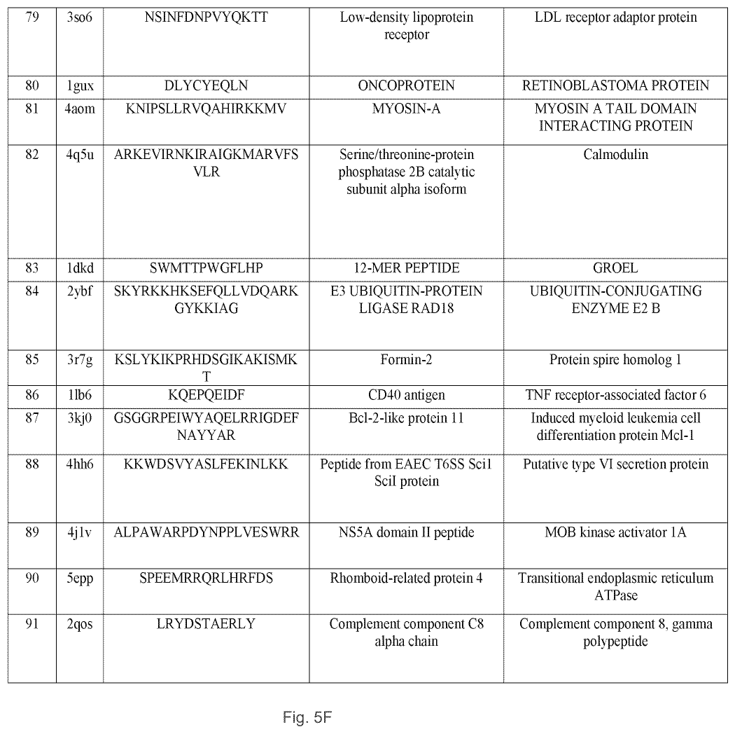

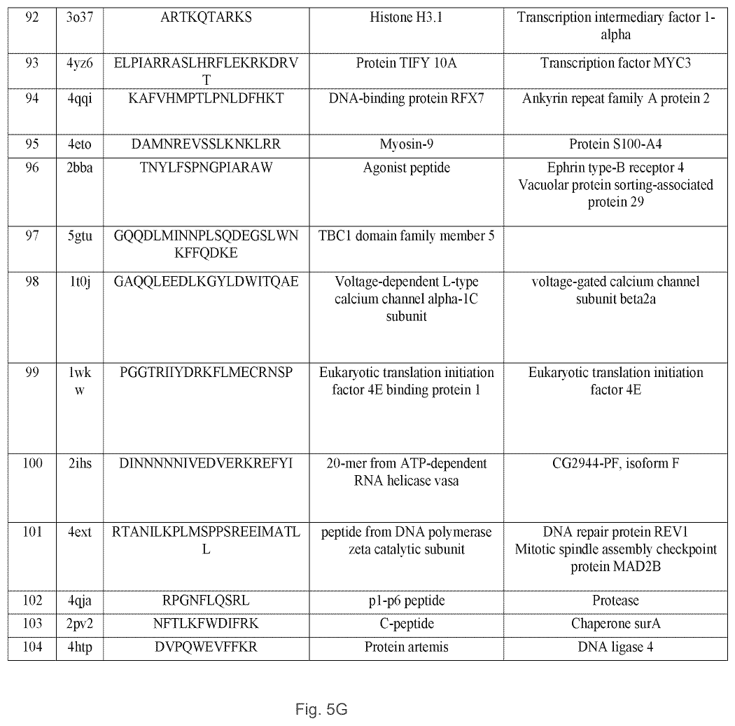

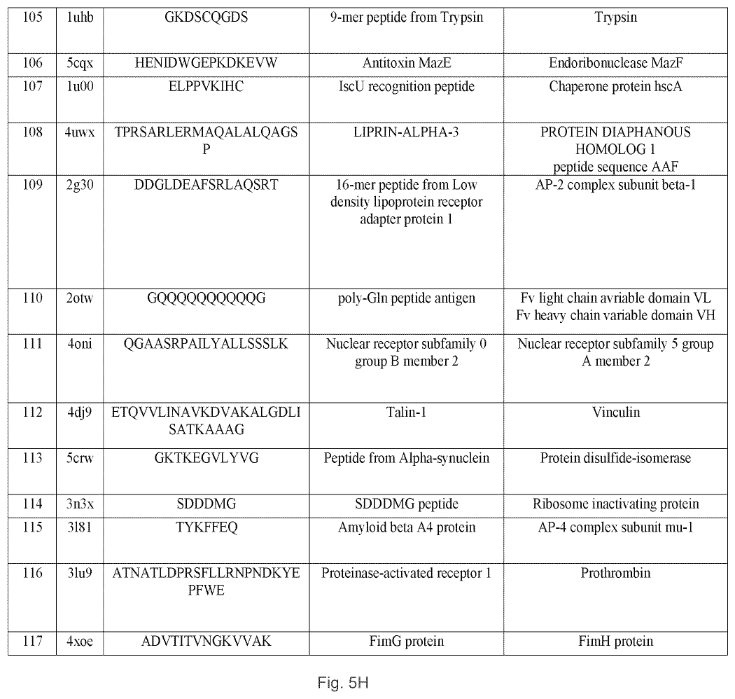

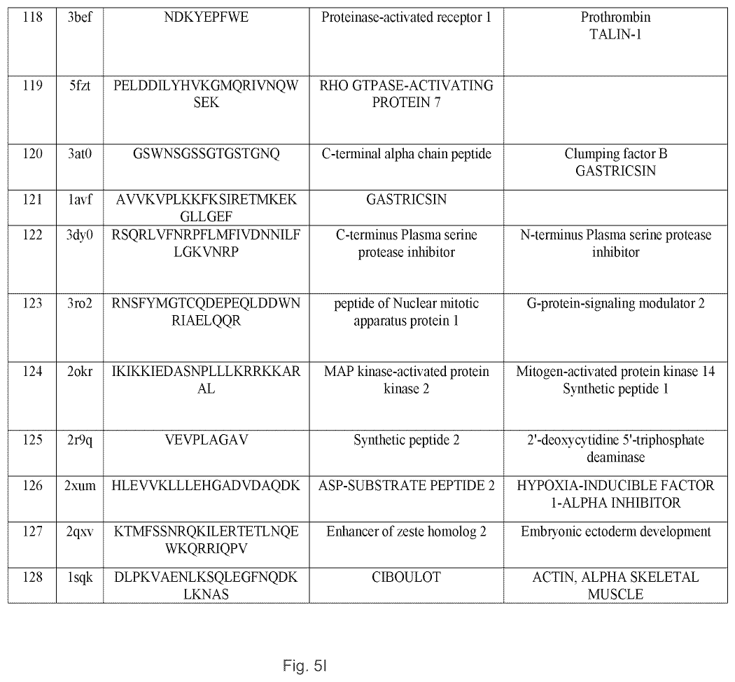

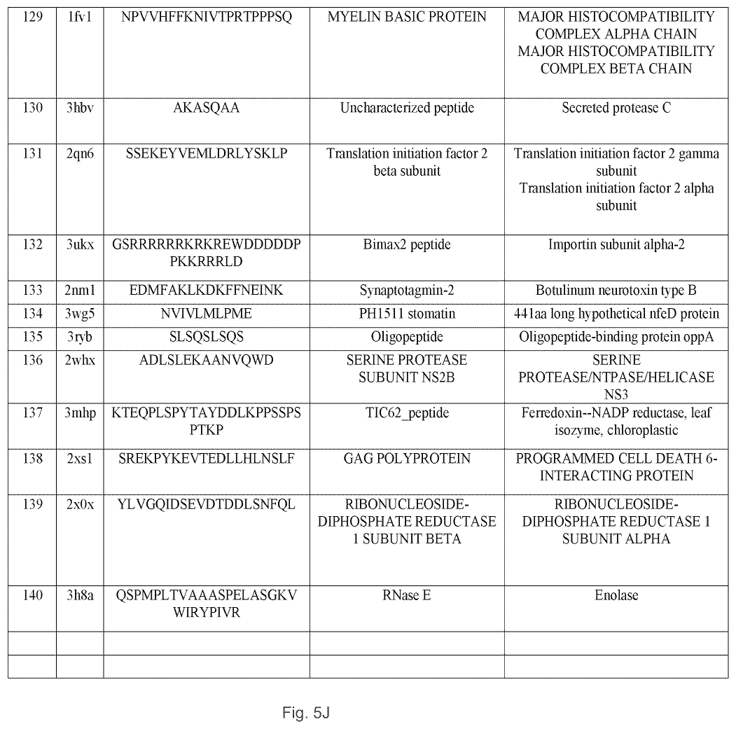

The results of this example are shown in FIG. 4.

A nitrocellulose membrane was printed with 7 lines:

The top line is 50 ng/μl of PG1-GFP-SH3 molecule log 10 dilution of protein for each subsequent line. The bottom line is 5×10-5. The more lines visible the higher the signal amplification. SH3 and MBDIP are protein tag binding partners. PG1 is protein G which binds to IgG

### Protocol

PBS 1% tween running buffer is run up the strip from the sample pad until it reaches the absorbent pad at the top of the nitrocellulose membrane (NC). 1 ul of each component is then added at the bottom of the NC. Strips are placed back into running buffer solution for 5 minutes to wash.

Repeat steps 2 and 3 until all components have been added.

Strips 1 & 2. (from different set of control strips imaged separately)

### Components:

1. Anti-biotin gold (commercially available from BBI, IgG in antibody binds in 1 to 1 interaction to PG1 site of printed protein)

### Strips 3 & 4.

### Components:

1. Biotinylated MbDIP protein
2. Anti-biotin gold

Each SH3 on the immobilised PG1-GFP-SH3 binds to a single biotinylated MBDIP protein having multiple biotin labels. Each biotin picks up a gold particle. This approach provides a limited means of amplification, similar to known art methods.

### Strips 5 & 6

### Components:

1. T7 bacteriophage with MbDIP protein in gene 10
2. T7 bacteriophage with SH3 protein in gene 10
3. Biotinylated MBDIP protein
4. Anti-biotin gold

Each immobilised SH3 on the immobilised PG1-GFP-SH3 picks up a capsid MbDIP protein having multiple free MbDIP sites. Each free MbDIP can bid to a capsid having multiple SH3 sites. Thus the single immobilised SH3 becomes many. Each SH3 binds to a single biotinylated MbDIP protein having multiple biotin labels. Each biotin picks up a gold particle. Signal amplification therefore occurs due to the multiple constructs, each having repeating affinity sites (herein SH3 and MbDIP).

Results seen in FIG. 4 show that lines 5 and 6 (having more particles per construct) can be seen at a lower concentration than lines 3 and 4, which are more sensitive than lanes 1 and 2 having a single nanoparticle per construct.

1.-10. (canceled)

11. A flow device for detecting the presence of a target, the device comprising:
(a) a sample loading area,
(b1) an area comprising a first labelled capsid comprising a region that is specific for a target wherein the first labelled capsid is capable of wicking across at least a portion of the lateral flow device,
(c) an area specific for the detectable marker;
(d) an area comprising capture probes for the specific target, and optionally a separate area comprising capture probes specific for the first labelled capsid; wherein said capture probes are immobilized on the lateral flow device; and
(d) absorbent material, wherein the absorbent material wicks an aqueous sample across the lateral flow device when the aqueous sample is added to the sample loading area.

12. The flow device according to claim 11 comprising a further area (b2) comprising a second labelled capsid configured to bind the first labelled capsid through tag/binder interactions wherein the second labelled capsid is capable of wicking across at least a portion of the lateral flow device.

13. A method for detecting the presence of a target in a sample comprising:
i) adding the sample to a sample loading area of a flow device according to claim 11; and
ii) determining the presence of the target in the sample from the presence of detectably labelled probe in (c) of the flow device.

14. A kit of reagents for detecting a target, the kit comprising a device as claimed in claim 11 and a buffer solution into which a biological sample can be added and optionally instructions for use of the kit.

15. The kit of claim 14, wherein the flow device is provided sterile in a closed and sealed packaging.

16. The flow device according to claim 11, wherein the first labelled capsid comprises a plurality of viral coat proteins wherein at least a proportion of the coat proteins comprise a first affinity peptide that comprises multiple copies of a first affinity tag specific for a protein or peptide binding partner, wherein the capsid additionally comprises a region specific for a target.

17. The flow device according to claim 12, wherein the second labelled capsid comprises a plurality of viral coat proteins, wherein at least a first proportion of the coat proteins carry a binding partner specific for the first affinity tags present on the first labelled capsid and a second proportion of the coat proteins carry a second affinity peptide that comprises multiple copies of a second affinity tag.

18. The flow device according to claim 12, wherein the second labelled capsid is configured to bind the first labelled capsid through tag/binder interactions between the affinity tags on the first labelled capsid and the binding partner on the second labelled capsid.

19. The flow device according to claim 16, wherein the first affinity tag is an epitope tag, or a non-antibody protein or peptide binding tag.

20. The flow device according to claim 17, wherein the second affinity tag is an epitope tag, or a non-antibody protein or peptide binding tag.

21. The Dow device according to claim 16, wherein the first affinity peptide is attached to the N terminus or C terminus of the capsid proteins.

22. The flow device according to claim 16, wherein the protein or peptide binding partner is attached to the N terminus or C terminus of the capsid proteins.

23. The flow device according to claim 16, wherein the first affinity peptide is attached to the N terminus or C terminus of the capsid proteins.

24. The flow device according to claim 16, wherein the protein or peptide binding partner is attached to the N terminus or C terminus of the capsid proteins.

25. The flow device according to claim 16, wherein the region specific for a target is selected from a nucleotide sequence which is complementary or partially complementary to a DNA sequence or RNA sequence target; an antibody or a target binding fragment thereof, a non-antibody protein which is specific for its cognate binding partner, where the cognate binding partner is the target, or cognate binding partner, where the non-antibody protein is the target.

26. The flow device according to claim 11, wherein the first labelled capsid comprises a multi protein assembly of capsid proteins, the multi protein assembly comprising a plurality of units, said units being exclusively one of:
(i) a peptide comprising repeating amino acid sequences which are affinity tags for specific binding partners; or
(ii) the specific binding partner for an affinity tag:
wherein the first labelled capsid additionally comprises a region specific for a target in a sample.

27. The flow device according to claim 12, wherein the second labelled capsid comprises a multi protein assembly of capsid proteins, the multi protein assembly comprising two different units, each unit being one of:
(a) capsid proteins that comprises a peptide that comprises at least two repeats of a peptide affinity tag specific for a peptide or protein binding partner; or
(b) capsid proteins that comprise the peptide sequence of a peptide or protein binding partner specific for a peptide affinity tag,
wherein the affinity tag of (a) is configured to bind the binding partner of the first labelled capsid or the protein binding partner of (b) is configured to bind the affinity tag of the first labelled capsid, whichever is present.


SEQUENCE LISTING (140 sequences)

SEQ ID NO: 1 - protein, 13 aa, synthetic construct
  REGION 1..13: note = Alpha-Tag
SRLEEELRRRLTE

SEQ ID NO: 2 - protein, 4 aa, synthetic construct
  REGION 1..4: note = C-tag
EPEA

SEQ ID NO: 3 - protein, 26 aa, synthetic construct
  REGION 1..26: note = Calmodulin Tag
KRRWKKNFIAVSAANRFKKISSSGAL

SEQ ID NO: 4 - protein, 13 aa, synthetic construct
  REGION 1..13: note = E-tag
GAPVPYPDPLEPR

SEQ ID NO: 5 - protein, 8 aa, synthetic construct
  REGION 1..8: note = FLAG Tag
DYKDDDDK

SEQ ID NO: 6 - protein, 19 aa, synthetic construct
  REGION 1..19: note = G4T Tag
EELLSKNYHLENEVARLKK

SEQ ID NO: 7 - protein, 8 aa, synthetic construct
  REGION 1..8: note = Glu-Glu tag
EEEEYMPE

SEQ ID NO: 8 - protein, 9 aa, synthetic construct
  REGION 1..9: note = HA Tag
YPYDVPDYA

SEQ ID NO: 9 - protein, 10 aa, synthetic construct
  REGION 1..10: note = Myc Tag
EQKLISEEDL

SEQ ID NO: 10 - protein, 18 aa, synthetic construct
  REGION 1..18: note = NE Tag
TKENPRSNQEESYDDNES

SEQ ID NO: 11 - protein, 9 aa, synthetic construct
  REGION 1..9: note = Rho1D4 Tag
TETSQVAPA

SEQ ID NO: 12 - protein, 13 aa, synthetic construct
  REGION 1..13: note = Softag 1
SLAELLNAGLGGS

SEQ ID NO: 13 - protein, 8 aa, synthetic construct
  REGION 1..8: note = Softag 3
TQDPSRVG

SEQ ID NO: 14 - protein, 12 aa, synthetic construct
  REGION 1..12: note = Spot Tag
PDRVRAVSHWSS

SEQ ID NO: 15 - protein, 15 aa, synthetic construct
  REGION 1..15: note = S-Tag
KETAAAKFERQHMDS

SEQ ID NO: 16 - protein, 11 aa, synthetic construct
  REGION 1..11: note = T7tag
MASMTGGQQMG

SEQ ID NO: 17 - protein, 13 aa, synthetic construct
  REGION 1..13: note = EG2-tag
RQLINTNGSWHIN

SEQ ID NO: 18 - protein, 10 aa, synthetic construct
  REGION 1..10: note = BC2 Peptide
PDRKAAVSHW

SEQ ID NO: 19 - protein, 10 aa, synthetic construct
  REGION 1..10: note = TC Tag
EVHTNQDPLD

SEQ ID NO: 20 - protein, 11 aa, synthetic construct
  REGION 1..11: note = VSV Tag
YTDIEMNRLGK

SEQ ID NO: 21 - protein, 8 aa, synthetic construct
  REGION 1..8: note = Xpress Tag
DLYDDDDK

SEQ ID NO: 22 - protein, 15 aa, synthetic construct
  REGION 1..15: note = Avi-tag
GLNDIFEAQKIEWHE

SEQ ID NO: 23 - protein, 23 aa, synthetic construct
  REGION 1..23: note = DogTag
DIPATYEFTDGKHYITNEPIPPK

SEQ ID NO: 24 - protein, 16 aa, synthetic construct
  REGION 1..16: note = Isopeptag
TDKDMTITFTNKKDAE

SEQ ID NO: 25 - protein, 38 aa, synthetic construct
  REGION 1..38: note = SBP Tag
MDEKTTGWRGGHVVEGLAGELEQLRARLEHHPQGQREP

SEQ ID NO: 26 - protein, 13 aa, synthetic construct
  REGION 1..13: note = Sdytag
DPIVMIDNDKPIT

SEQ ID NO: 27 - protein, 13 aa, synthetic construct
  REGION 1..13: note = SH3 Tag
STVPVAPPRRRRG

SEQ ID NO: 28 - protein, 12 aa, synthetic construct
  REGION 1..12: note = Snooptag
KLGDIEFIKVNK

SEQ ID NO: 29 - protein, 13 aa, synthetic construct
  REGION 1..13: note = Spytag
AHIVMVDAYKPTK

SEQ ID NO: 30 - protein, 8 aa, synthetic construct
  REGION 1..8: note = Strept Tag
WSHPQFEK

SEQ ID NO: 31 - protein, 81 aa, synthetic construct
  REGION 1..81: note = MbDIP
GRLDLPPGFMFKVQAQHDYTATDTDELQLKAGDVVLVIPFQNPEEQDEGWLMGVKESDWNQHKELEKCRGVFPENFTERVP

SEQ ID NO: 32 - protein, 7 aa, synthetic construct
  REGION 1..7: note = Poly Glutamate Tag
EEEEEEE

SEQ ID NO: 33 - protein, 7 aa, synthetic construct
  REGION 1..7: note = Poly Arginine Tag
RRRRRRR

SEQ ID NO: 34 - protein, 6 aa, synthetic construct
  REGION 1..6: note = His Tag
HHHHHH

SEQ ID NO: 35 - protein, 6 aa, synthetic construct
  REGION 1..6: note = Ty Tag
CCPGCC

SEQ ID NO: 36 - protein, 5 aa, Escherichia coli
VDAVN

SEQ ID NO: 37 - protein, 7 aa, Alphapapillomavirus 7
  source 1..7: note = Human papillomavirus type 18
RRRETQV

SEQ ID NO: 38 - protein, 11 aa, Homo sapiens
GMRPPPPGIRG

SEQ ID NO: 39 - protein, 8 aa, synthetic construct
  REGION 1..8: note = APP-tag
APPLPPRN

SEQ ID NO: 40 - protein, 10 aa, synthetic construct
  REGION 1..10: note = iCAL-tag
ANSRFPTSII

SEQ ID NO: 41 - protein, 7 aa, synthetic construct
  REGION 1..7: note = ACE-tag
PWATCDS

SEQ ID NO: 42 - protein, 8 aa, Homo sapiens
DAEFRHDS

SEQ ID NO: 43 - protein, 7 aa, synthetic construct
  REGION 1..7: note = BAX-tag
PTSSEQI

SEQ ID NO: 44 - protein, 8 aa, Homo sapiens
ENQKEYFF

SEQ ID NO: 45 - protein, 9 aa, Human immunodeficiency virus 1
LELDKWASL

SEQ ID NO: 46 - protein, 12 aa, synthetic construct
  REGION 1..12: note = H3-tag
DKQVEYLDLDLD

SEQ ID NO: 47 - protein, 11 aa, synthetic construct
  REGION 1..11: note = H3-tag
ARTKQTARKST

SEQ ID NO: 48 - protein, 9 aa, synthetic construct
  REGION 1..9: note = TAT-tag
PKLEPWKHP

SEQ ID NO: 49 - protein, 8 aa, Homo sapiens
ITIPVTFE

SEQ ID NO: 50 - protein, 10 aa, Homo sapiens
SDATEGHDED

SEQ ID NO: 51 - protein, 12 aa, Homo sapiens
TPEAPPCYMDVI

SEQ ID NO: 52 - protein, 15 aa, synthetic construct
  REGION 1..15: note = OuD-tag
RTFRQVQSSISDFYD

SEQ ID NO: 53 - protein, 16 aa, Mus musculus
GQPPLVPPRKEKMRGK

SEQ ID NO: 54 - protein, 9 aa, Homo sapiens
DSGFSFGSK

SEQ ID NO: 55 - protein, 10 aa, synthetic construct
  REGION 1..10: note = Tag 1
ARTKQTARKS

SEQ ID NO: 56 - protein, 18 aa, synthetic construct
  REGION 1..18: note = Tag 2
SEADEMAKALEAELNDLM

SEQ ID NO: 57 - protein, 13 aa, Rattus norvegicus
GPRLSRLLSSAGC

SEQ ID NO: 58 - protein, 13 aa, synthetic construct
  REGION 1..13: note = Tag 3
WRYYESSLLPYPD

SEQ ID NO: 59 - protein, 9 aa, Mus musculus
RIPSYRYRY

SEQ ID NO: 60 - protein, 21 aa, Saccharomyces cerevisiae
SLSIEETNELRASLGLKLIPP

SEQ ID NO: 61 - protein, 17 aa, Homo sapiens
SNLNPNAAEFVPGVKYG

SEQ ID NO: 62 - protein, 10 aa, Homo sapiens
GERTIPITRE

SEQ ID NO: 63 - protein, 15 aa, synthetic construct
  REGION 1..15: note = Tag 4
SQETFSDLWKLLPEN

SEQ ID NO: 64 - protein, 22 aa, Homo sapiens
MDEQEALNSIMNDLVALQMNRR

SEQ ID NO: 65 - protein, 24 aa, Saccharomyces cerevisiae
KSLRVSSLNKDRRLLLREFYNLEN

SEQ ID NO: 66 - protein, 18 aa, synthetic construct
  REGION 1..18: note = Tag 5
GQVTTESNLVEFDEESTK

SEQ ID NO: 67 - protein, 6 aa, synthetic construct
  REGION 1..6: note = Tag 6
AVPIAQ

SEQ ID NO: 68 - protein, 19 aa, Drosophila melanogaster
GPGSRGKSTVTGRMISGWL

SEQ ID NO: 69 - protein, 5 aa, synthetic construct
  REGION 1..5: note = Tag 7
DDLYG

SEQ ID NO: 70 - protein, 7 aa, synthetic construct
  REGION 1..7: note = Tag 8
WEYIPNV

SEQ ID NO: 71 - protein, 11 aa, synthetic construct
  REGION 1..11: note = Tag 9
KSLTIYAQVQK

SEQ ID NO: 72 - protein, 10 aa, synthetic construct
  REGION 1..10: note = Tag 10
KRKSRWDETP

SEQ ID NO: 73 - protein, 14 aa, Homo sapiens
LPPTEQDLTKLLLE

SEQ ID NO: 74 - protein, 9 aa, synthetic construct
  REGION 1..9: note = Tag 11
PQIINRPQN

SEQ ID NO: 75 - protein, 11 aa, Homo sapiens
PARPPKPRPRR

SEQ ID NO: 76 - protein, 18 aa, synthetic construct
  REGION 1..18: note = Tag 12
AYQQGQNQLYNELNLGRR

SEQ ID NO: 77 - protein, 5 aa, synthetic construct
  REGION 1..5: note = Tag 13
VAFRS

SEQ ID NO: 78 - protein, 16 aa, synthetic construct
  REGION 1..16: note = Tag 14
MGDSILSQAEIDALLN

SEQ ID NO: 79 - protein, 14 aa, Homo sapiens
NSINFDNPVYQKTT

SEQ ID NO: 80 - protein, 9 aa, unidentified
  source 1..9: note = Human papillomavirus
DLYCYEQLN

SEQ ID NO: 81 - protein, 18 aa, unidentified
  source 1..18: note = Plasmodium falciparum
KNIPSLLRVQAHIRKKMV

SEQ ID NO: 82 - protein, 24 aa, Homo sapiens
ARKEVIRNKIRAIGKMARVFSVLR

SEQ ID NO: 83 - protein, 12 aa, synthetic construct
  REGION 1..12: note = Tag 15
SWMTTPWGFLHP

SEQ ID NO: 84 - protein, 27 aa, Homo sapiens
SKYRKKHKSEFQLLVDQARKGYKKIAG

SEQ ID NO: 85 - protein, 22 aa, Homo sapiens
KSLYKIKPRHDSGIKAKISMKT

SEQ ID NO: 86 - protein, 9 aa, Homo sapiens
KQEPQEIDF

SEQ ID NO: 87 - protein, 27 aa, Homo sapiens
GSGGRPEIWYAQELRRIGDEFNAYYAR

SEQ ID NO: 88 - protein, 18 aa, synthetic construct
  REGION 1..18: note = Tag 16
KKWDSVYASLFEKINLKK

SEQ ID NO: 89 - protein, 20 aa, Hepacivirus C
  source 1..20: note = Hepatitis C virus
ALPAWARPDYNPPLVESWRR

SEQ ID NO: 90 - protein, 15 aa, Homo sapiens
SPEEMRRQRLHRFDS

SEQ ID NO: 91 - protein, 11 aa, synthetic construct
  REGION 1..11: note = Tag 17
LRYDSTAERLY

SEQ ID NO: 92 - protein, 10 aa, Homo sapiens
ARTKQTARKS

SEQ ID NO: 93 - protein, 22 aa, Arabidopsis thaliana
ELPIARRASLHRFLEKRKDRVT

SEQ ID NO: 94 - protein, 17 aa, Homo sapiens
KAFVHMPTLPNLDFHKT

SEQ ID NO: 95 - protein, 16 aa, Homo sapiens
DAMNREVSSLKNKLRR

SEQ ID NO: 96 - protein, 15 aa, synthetic construct
  REGION 1..15: note = Tag 18
TNYLFSPNGPIARAW

SEQ ID NO: 97 - protein, 27 aa, Homo sapiens
GQQDLMINNPLSQDEGSLWNKFFQDKE

SEQ ID NO: 98 - protein, 20 aa, Homo sapiens
GAQQLEEDLKGYLDWITQAE

SEQ ID NO: 99 - protein, 20 aa, Homo sapiens
PGGTRIIYDRKFLMECRNSP

SEQ ID NO: 100 - protein, 20 aa, Drosophila melanogaster
DINNNNNIVEDVERKREFYI

SEQ ID NO: 101 - protein, 23 aa, Homo sapiens
RTANILKPLMSPPSREEIMATLL

SEQ ID NO: 102 - protein, 10 aa, Human immunodeficiency virus 1
  source 1..10: note = Human immunodeficiency virus type 1
RPGNFLQSRL

SEQ ID NO: 103 - protein, 12 aa, synthetic construct
  REGION 1..12: note = Tag 19
NFTLKFWDIFRK

SEQ ID NO: 104 - protein, 11 aa, Homo sapiens
DVPQWEVFFKR

SEQ ID NO: 105 - protein, 9 aa, Sus scrofa
GKDSCQGDS

SEQ ID NO: 106 - protein, 15 aa, Escherichia coli
HENIDWGEPKDKEVW

SEQ ID NO: 107 - protein, 9 aa, synthetic construct
  REGION 1..9: note = Tag 20
ELPPVKIHC

SEQ ID NO: 108 - protein, 21 aa, Mus musculus
TPRSARLERMAQALALQAGSP

SEQ ID NO: 109 - protein, 16 aa, synthetic construct
  REGION 1..16: note = Tag 21
DDGLDEAFSRLAQSRT

SEQ ID NO: 110 - protein, 12 aa, synthetic construct
  REGION 1..12: note = Tag 22
GQQQQQQQQQQG

SEQ ID NO: 111 - protein, 19 aa, Homo sapiens
QGAASRPAILYALLSSSLK

SEQ ID NO: 112 - protein, 29 aa, Homo sapiens
ETQVVLINAVKDVAKALGDLISATKAAAG

SEQ ID NO: 113 - protein, 11 aa, Homo sapiens
GKTKEGVLYVG

SEQ ID NO: 114 - protein, 6 aa, synthetic construct
  REGION 1..6: note = Tag 23
SDDDMG

SEQ ID NO: 115 - protein, 7 aa, synthetic construct
  REGION 1..7: note = Tag 24
TYKFFEQ

SEQ ID NO: 116 - protein, 25 aa, Homo sapiens
ATNATLDPRSFLLRNPNDKYEPFWE

SEQ ID NO: 117 - protein, 14 aa, Escherichia coli
ADVTITVNGKVVAK

SEQ ID NO: 118 - protein, 9 aa, Homo sapiens
NDKYEPFWE

SEQ ID NO: 119 - protein, 23 aa, Homo sapiens
PELDDILYHVKGMQRIVNQWSEK

SEQ ID NO: 120 - protein, 16 aa, Homo sapiens
GSWNSGSSGTGSTGNQ

SEQ ID NO: 121 - protein, 26 aa, Homo sapiens
AVVKVPLKKFKSIRETMKEKGLLGEF

SEQ ID NO: 122 - protein, 29 aa, Homo sapiens
RSQRLVFNRPFLMFIVDNNILFLGKVNRP

SEQ ID NO: 123 - protein, 28 aa, Homo sapiens
RNSFYMGTCQDEPEQLDDWNRIAELQQR

SEQ ID NO: 124 - protein, 24 aa, synthetic construct
  REGION 1..24: note = Tag 25
IKIKKIEDASNPLLLKRRKKARAL

SEQ ID NO: 125 - protein, 9 aa, synthetic construct
  REGION 1..9: note = Tag 26
VEVPLAGAV

SEQ ID NO: 126 - protein, 20 aa, synthetic construct
  REGION 1..20: note = Tag 27
HLEVVKLLLEHGADVDAQDK

SEQ ID NO: 127 - protein, 30 aa, Mus musculus
KTMFSSNRQKILERTETLNQEWKQRRIQPV

SEQ ID NO: 128 - protein, 25 aa, Drosophila melanogaster
DLPKVAENLKSQLEGFNQDKLKNAS

SEQ ID NO: 129 - protein, 20 aa, synthetic construct
  REGION 1..20: note = Tag 28
NPVVHFFKNIVTPRTPPPSQ

SEQ ID NO: 130 - protein, 7 aa, synthetic construct
  REGION 1..7: note = Tag 29
AKASQAA

SEQ ID NO: 131 - protein, 18 aa, synthetic construct
  REGION 1..18: note = Tag 30
SSEKEYVEMLDRLYSKLP

SEQ ID NO: 132 - protein, 28 aa, synthetic construct
  REGION 1..28: note = Tag 31
GSRRRRRRKRKREWDDDDDPPKKRRRLD

SEQ ID NO: 133 - protein, 17 aa, synthetic construct
  REGION 1..17: note = Tag 32
EDMFAKLKDKFFNEINK

SEQ ID NO: 134 - protein, 10 aa, Pyrococcus horikoshii
NVIVLMLPME

SEQ ID NO: 135 - protein, 9 aa, synthetic construct
  REGION 1..9: note = Tag 33
SLSQSLSQS

SEQ ID NO: 136 - protein, 14 aa, Dengue virus
  source 1..14: note = Dengue virus type 4
ADLSLEKAANVQWD

SEQ ID NO: 137 - protein, 26 aa, synthetic construct
  REGION 1..26: note = Tag 34
KTEQPLSPYTAYDDLKPPSSPSPTKP

SEQ ID NO: 138 - protein, 20 aa, Simian immunodeficiency virus
SREKPYKEVTEDLLHLNSLF

SEQ ID NO: 139 - protein, 20 aa, Escherichia coli
YLVGQIDSEVDTDDLSNFQL

SEQ ID NO: 140 - protein, 28 aa, Escherichia coli
QSPMPLTVAAASPELASGKVWIRYPIVR